(19) United States
(12) Patent Application Publication
(10) Pub. No.: US 2026/0060637 A1
(43) Pub. Date: Mar. 5, 2026

(54) SYSTEMS FOR REAL-TIME DETECTION OF RETURN OF SPONTANEOUS CIRCULATION USING DOPPLER ULTRASOUND AND METHODS OF USE THEREOF

(71) Applicant: THE FEINSTEIN INSTITUTES FOR MEDICAL RESEARCH, INC., Manhasset, NY (US)

(72) Inventors: Daniel ROLSTON, Manhasset, NY (US); Lance BECKER, Manhasset, NY (US); Daniel JAFARI, Manhasset, NY (US); Allison COHEN, Manhasset, NY (US); Timmy LI, Manhasset, NY (US)

(21) Appl. No.: 19/314,058

(22) Filed: Aug. 29, 2025

Related U.S. Application Data
(60) Provisional application No. 63/690,032, filed on Sep. 3, 2024.

(51) Int. Cl. A61B 8/06; A61B 8/00

(52) U.S. Cl. CPC A61B 8/06; A61B 8/403; A61B 8/4236; A61B 8/461; A61B 8/488

(57) ABSTRACT

A system and method provide real-time detection of return of spontaneous circulation (ROSC) during cardiac arrest using at least one Doppler ultrasound patch with a pulsed-wave or continuous wave ultrasound transducer that emits an ultrasound beam into a subject's skin over a carotid, femoral, or brachial artery and receives at least one Doppler return signal indicative of blood flow. A fixation mechanism secures the transducer in a fixed position relative to the carotid, femoral, brachial artery, and a wired or wireless communication interface transmits the Doppler return signal to a processing unit. The processing unit receives and analyzes the Doppler return signal using a Doppler-Integrated Cardiac Arrest Flow Analysis (DICAF) algorithm, which uses a machine learning model trained on annotated Doppler waveform datasets to detect a first blood flow pattern generated by chest compressions and a second blood flow pattern generated by intrinsic cardiac activity. The system determines ROSC in real time and generates an alert for the user. The system can also monitor the quality of blood flow generated by chest compressions to guide cardiopulmonary resuscitation.

### FIELD OF DISCLOSURE

The present disclosure generally relates to medical technologies, and more specifically to systems for real-time detection of return of spontaneous circulation (ROSC) during cardiac arrest using Doppler ultrasound and methods of use thereof.

### BACKGROUND OF THE DISCLOSURE

Cardiac arrest is a significant cause of mortality worldwide, particularly in out-of-hospital settings where prompt and effective cardiopulmonary resuscitation (CPR) may not be readily available. Even within hospital environments, timely detection of return of spontaneous circulation (ROSC) and rapid initiation of post-resuscitation care are critical for improving patient outcomes. Current guidelines for ROSC detection typically require pausing CPR every two minutes for a pulse check, which is highly dependent on the skill of the practitioner and is known to have poor overall accuracy and low sensitivity when a pulse is present. This can result in delays in administering life-saving interventions and post-resuscitation care, as well as inaccurate estimations of cardiac arrest duration, which may negatively influence clinical decision-making.

Frequent and prolonged interruptions for pulse checks also reduce the chest compression fraction (CCF)—the proportion of time during which compressions are performed—which is a key determinant of survival in cardiac arrest patients. Furthermore, a substantial proportion of patients who initially achieve ROSC may experience re-arrest within minutes, and delays in recognizing recurrent cardiac arrest are common.

Traditional methods for ROSC detection, such as manual pulse checks, are time-consuming, subject to high inter-operator variability, and lack specificity. Studies have shown that a significant percentage of healthcare providers are unable to accurately detect a carotid pulse during cardiac arrest scenarios. While invasive arterial lines can provide continuous monitoring, their placement is time-consuming and often impractical in pre-hospital or many emergency department settings. Non-invasive alternatives, such as Doppler ultrasound, have shown promise for monitoring blood flow, but existing approaches typically require pausing compressions, manual operation, and expert interpretation. Recent developments in hands-free ultrasound devices are still in early stages and have not yet achieved widespread clinical adoption.

Accordingly, there remains a need for improved systems and methods that enable continuous, reliable, and non-invasive monitoring of blood flow during cardiac arrest. Such systems should facilitate real-time detection of ROSC and provide actionable feedback to optimize CPR quality, without requiring interruptions in chest compressions or specialized operator expertise.

### SUMMARY

An illustrative system may include at least one Doppler ultrasound patch including a pulsed wave or continuous wave ultrasound transducer that may be configured to emit an ultrasound beam into a selected artery in a neck, a groin/upper leg, or an arm of a subject over a carotid, a femoral, or a brachial artery and may be configured to receive at least one Doppler return signal indicative of blood flow in the selected artery. The system may further include a fixation mechanism that may be configured to secure the pulsed wave or continuous wave ultrasound transducer in a fixed position relative to the selected artery, and a wireless or wired communication interface that may be configured to transmit the at least one Doppler return signal to a processing unit. The processing unit may include at least one transitory memory storing at least one instruction and at least one processor, which may be configured to execute the at least one instruction that may cause the at least one processor to receive the at least one Doppler return signal from the at least one Doppler ultrasound patch, analyze the at least one Doppler return signal indicative of the blood flow in the selected artery using a Doppler-Integrated Cardiac Arrest Flow Analysis (DICAF) algorithm to detect during ongoing chest compressions a first blood flow pattern including a bidirectional flow or minimal blood flow pattern corresponding to the blood flow generated by the ongoing chest compressions and a second blood flow pattern corresponding to intrinsic cardiac activity originating from within a heart of the subject in addition to or altering the blood flow generated by chest compressions. The DICAF algorithm may include at least one machine learning model trained on annotated Doppler waveform datasets to distinguish between the blood flow generated by the ongoing chest compressions and the blood flow associated with the intrinsic cardiac activity in the first blood flow pattern and the second blood flow pattern. The second blood flow pattern may include at least one of systolic pulses generated by the heart superimposed on the first blood flow pattern or a predominance of an anterograde flow level relative to a retrograde flow level. The processing unit may determine in real time during the ongoing chest compressions a return of spontaneous circulation based on detection of the second blood flow pattern and may generate an alert including an indication of the return of spontaneous circulation or a recommendation to continue applying the ongoing chest compressions to the subject due to a lack of the return of spontaneous circulation, and may include an output interface to display the alert.

A method may include receiving, by at least one processor, at least one Doppler return signal from at least one Doppler ultrasound patch positioned over a selected artery of a subject. The method may further include analyzing, by the at least one processor, the at least one Doppler return signal indicative of blood flow in the selected artery using a Doppler-Integrated Cardiac Arrest Flow Analysis (DICAF) algorithm to detect during ongoing chest compressions a first blood flow pattern including a bidirectional flow corresponding to the blood flow generated by the ongoing chest compressions and a second blood flow pattern corresponding to intrinsic cardiac activity originating from within a heart of the subject. The DICAF algorithm may include at least one machine learning model trained on annotated Doppler waveform datasets to distinguish between the blood flow generated by the ongoing chest compressions and the blood flow associated with the intrinsic cardiac activity in the first blood flow pattern and the second blood flow pattern. The second blood flow pattern may include at least one of systolic pulses generated by the heart superimposed on the first blood flow pattern or a predominance of an anterograde flow value relative to a retrograde flow value. The method may further include determining, by the at least one processor, in real time during the ongoing chest compressions, a return of spontaneous circulation based on detection of the second blood flow pattern, generating, by the at least one processor, an alert including an indication of the return of spontaneous circulation or a recommendation to continue applying the ongoing chest compressions to the subject due to a lack of the return of spontaneous circulation, and transmitting, by the at least one processor, at least one instruction to an output interface to display the alert.

### BRIEF DESCRIPTION OF THE DRAWINGS

Various embodiments of the present disclosure can be further explained with reference to the attached drawings, wherein like structures are referred to by like numerals throughout the several views. The drawings shown are not necessarily to scale, with emphasis instead generally being placed upon illustrating the principles of the present disclosure. Specific structural and functional details disclosed herein are not to be interpreted as limiting, but merely as a representative basis for teaching one skilled in the art to variously employ one or more illustrative embodiments.

FIG. 1 is a biological diagram illustrating cardiopulmonary resuscitation (CPR) being performed on a subject with real-time monitoring using a Doppler ultrasound patch and a processing unit displaying return of spontaneous circulation (ROSC) status in accordance with one or more embodiments of the present disclosure;

FIG. 2 is a block diagram illustrating an embodiment of an illustrative processing system for real-time analysis of Doppler ultrasound signals to detect ROSC in accordance with one or more embodiments of the present disclosure.

FIG. 3 is a biological diagram illustrating CPR with Doppler ultrasound analysis, showing real-time monitoring of blood flow in the carotid artery using a wearable patch and processing unit in accordance with one or more embodiments of the present disclosure;

FIG. 4 is a graph illustrating a Doppler blood flow waveform showing bidirectional flow during cardiac arrest in accordance with one or more embodiments of the present disclosure;

FIG. 5 is an ultrasound image illustrating a Doppler waveform depicting pulsatility through chest compressions in accordance with one or more embodiments of the present disclosure;

FIG. 6 is a waveform diagram illustrating anterograde-dominant blood flow during chest compressions in accordance with one or more embodiments of the present disclosure; and

FIG. 7 is a flowchart illustrating a method for Doppler signal analysis to detect ROSC and generate corresponding alerts in accordance with one or more embodiments of the present disclosure.

### DETAILED DESCRIPTION OF THE EMBODIMENTS

Various detailed embodiments of the present disclosure, taken in conjunction with the accompanying figures, are disclosed herein; however, it is to be understood that the disclosed embodiments are merely illustrative. In addition, each of the examples given in connection with the various embodiments of the present disclosure is intended to be illustrative, and not restrictive.

Throughout the specification, the following terms take the meanings explicitly associated herein, unless the context clearly dictates otherwise. The phrases “in one embodiment” and “In at least some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

It is understood that at least one aspect/functionality of various embodiments described herein can be performed in real-time and/or dynamically. As used herein, the term “real-time” is directed to an event/action that can occur instantaneously or almost instantaneously in time when another event/action has occurred. For example, the “real-time processing,” “real-time computation,” and “real-time execution” all pertain to the performance of a computation during the actual time that the related physical process (e.g., a user interacting with an application on a mobile device) occurs, in order that results of the computation can be used in guiding the physical process.

As used herein, the term “dynamically” and term “automatically,” and their logical and/or linguistic relatives and/or derivatives, mean that certain events and/or actions can be triggered and/or occur without any human intervention. In at least some embodiments, events and/or actions in accordance with the present disclosure can be in real-time and/or based on a predetermined periodicity of at least one of: nanosecond, several nanoseconds, millisecond, several milliseconds, second, several seconds, minute, several minutes, hourly, several hours, daily, several days, weekly, monthly, etc.

As used herein, the term “runtime” corresponds to any behavior that is dynamically determined during an execution of a software application or at least a portion of software application.

In at least some embodiments of the present disclosure relate to systems and methods for real-time detection of return of spontaneous circulation (ROSC) during cardiac arrest using Doppler ultrasound technology. Cardiac arrest is a critical medical emergency in which rapid and accurate assessment of circulatory status is essential for guiding resuscitation efforts and improving patient outcomes. Conventional approaches for ROSC detection, such as manual pulse checks or invasive arterial lines, may be limited by inaccuracy, operator dependence, and the need to interrupt chest compressions, which may reduce the effectiveness of cardiopulmonary resuscitation (CPR).

The disclosed technology addresses these limitations by providing a non-invasive, hands-free system that may continuously monitor blood flow in a selected artery of a subject during ongoing chest compressions. By leveraging Doppler ultrasound technology, the illustrative system may deliver real-time feedback on circulatory status without requiring pauses in CPR or specialized operator expertise. This approach may enable more reliable and timely detection of ROSC, reduce unnecessary interruptions in chest compressions, and support physiologic-guided resuscitation.

It should be noted that although at least some of the embodiments herein refer to the Doppler signal analysis to detect ROSC and generate corresponding alerts in the carotid artery in a neck region of the subject, this is merely for visual and conceptual clarity and not by way of limitation of the embodiments disclosed herein. Any suitable artery may be selected, for example, by a user (e.g., health care provider, emergency medical team member, good Samaritan, etc.) such as a carotid artery (neck), a femoral artery (leg), and/or a brachial artery (arm) to perform the Doppler signal analysis to detect ROSC and generate corresponding alerts of the subject as described herein.

In at least some embodiments, various systems, devices, methods and/or principles of the present disclosure may be utilized based at least in part on and/or in conjunction with any systems, devices, methods and/or principles found in U.S. Patent Application Publication No. US 2024/0041696, published on Feb. 8, 2024, which is incorporated by reference herein in their entirety, and/or the article “Femoral artery Doppler ultrasound is more accurate than manual palpation for pulse detection in cardiac arrest” by A. L. Cohen, et al, published in Resuscitation, Vol. 173, pp. 157-165, 2022, which is incorporated herein by reference in its entirety for all purposes, especially any disclosure and/or teaching of both publications that may be related to the manufacturing, operation, and/or use of the at least one Doppler ultrasound patch as disclosed herein.

In at least some embodiments of the disclosure, the illustrative system may include at least one Doppler ultrasound patch that may be configured to emit and receive ultrasound signals over the selected artery. The patch may be secured in a fixed position and may wirelessly transmit Doppler return signals to a processing unit for analysis. The processing unit may include at least one processor and memory storing instructions that may enable automated analysis of the Doppler signals to detect blood flow patterns associated with both chest compressions and intrinsic cardiac activity.

In at least some embodiments, the at least one Doppler ultrasound patch may be connected by a wire to a defibrillator and/or other Doppler ultrasound output display.

In at least some embodiments, the at least one Doppler ultrasound patch may be coupled wirelessly to a defibrillator and/or other Doppler ultrasound output display.

In at least some embodiments, the at least one Doppler ultrasound patch may use continuous Doppler ultrasound, pulsed-wave Doppler ultrasound, or both, which may be also referred to as Spectral Doppler when displayed over time.

In at least some embodiments, the at least one Doppler ultrasound patch may be placed over the carotid artery, the femoral artery, or brachial artery, or any other suitable artery to detect blood flow for cardiac arrest analysis as described herein.

In at least some embodiments, the illustrative system may utilize a Doppler-Integrated Cardiac Arrest Flow Analysis (DICAF) Algorithm to distinguish between bidirectional or minimal blood flow generated by chest compressions and blood flow patterns indicative of intrinsic cardiac function. The processing unit may determine, in real time, whether ROSC has occurred and may generate an alert to inform the user, thereby providing actionable feedback to optimize CPR quality and minimize delays in post-resuscitation care.

In at least some embodiments, the illustrative system may provide real-time detection of return of spontaneous circulation (ROSC) during ongoing chest compressions using Doppler ultrasound waveform analysis. The illustrative system may continuously and non-invasively monitor blood flow in the selected artery during chest compressions, with automated analysis of Doppler signals to distinguish between compression-generated bidirectional flow and cardiac activity associated with ROSC. This approach may eliminate the need to pause compressions for pulse checks, enabling uninterrupted resuscitation and immediate feedback, which is not described or suggested in the prior art.

In at least some embodiments, an automated algorithm—such as the Doppler-Integrated Cardiac Arrest Flow Analysis (DICAF) Algorithm—may analyze Doppler waveforms to detect ROSC-specific patterns, including superimposed systolic pulses or anterograde-dominant flow, using machine learning trained on annotated datasets. This algorithmic approach for identifying ROSC-specific Doppler patterns are not disclosed in existing technologies, and the use of machine learning for real-time pattern recognition may further distinguish the illustrative system from conventional technologies.

In at least some embodiments, the illustrative system may include a wearable, hands-free or wired Doppler ultrasound patch with a pulsed wave or continuous-wave Doppler transducer, secured over the carotid, femoral, or brachial artery with a patch, and a wireless communication interface for transmitting Doppler signals to a processing unit. The Doppler ultrasound patch may provide a wide beam for reliable insonation and adhesive fixation for hands-free operation, offering technical features that improve usability and workflow during resuscitation, which are not found in prior art systems.

Note that the Doppler ultrasound patch may also be referred to herein as a Doppler ultrasound transducer patch, an ultrasound transducer patch, an ultrasound transducer and these terms may be used interchangeably.

In at least some embodiments, the illustrative system may generate actionable feedback and alerts for cardiopulmonary resuscitation (CPR) guidance, automatically indicating ROSC or recommending continuation of chest compressions based on real-time Doppler analysis. This real-time feedback may minimize dependence on manual checks and enhance the resuscitation process, introducing a new approach compared to existing systems that do not provide such targeted, automated alerts for ROSC recognition.

In at least some embodiments, the illustrative system may enable personalization of CPR based on real-time Doppler feedback, allowing assessment of chest compression efficacy by measuring the amount of blood flow generated and adjustment of compression location or technique to optimize blood flow. This physiologic-guided approach may tailor resuscitation to individual patient physiology, representing a meaningful improvement over existing techniques that do not use Doppler feedback for personalized CPR.

In at least some embodiments, the illustrative system may support the creation and use of Doppler waveform datasets from both cardiac arrest and non-arrest subjects to train machine learning algorithms for ROSC detection. The development of annotated datasets for algorithm training may enhance algorithm performance and validation, distinguishing this methodology from current technologies and supporting more accurate and reliable ROSC detection.

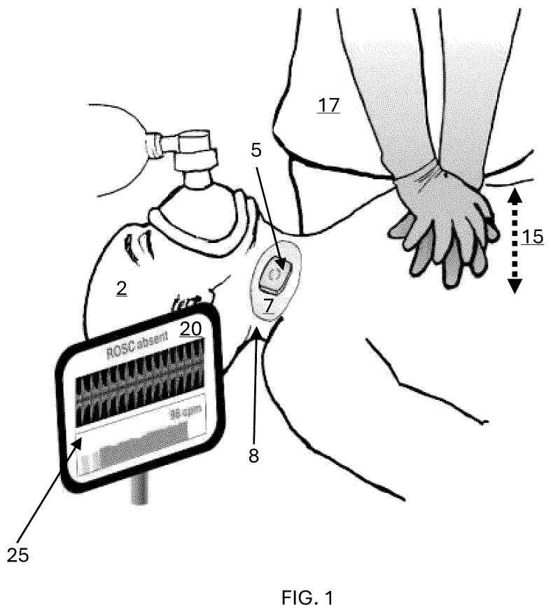

FIG. 1 is a biological diagram illustrating cardiopulmonary resuscitation (CPR) being performed on a subject 2 with real-time monitoring using a Doppler ultrasound patch 7 and a processing unit 20 displaying return of spontaneous circulation (ROSC) status in accordance with one or more embodiments of the present disclosure. Here, the figure may demonstrate the integration of various components of the illustrative system to enable continuous, non-invasive blood flow analysis during ongoing chest compressions 15.

In at least some embodiments, a Doppler Ultrasound Transducer Device 5 may be positioned over the region of the subject's neck 8, specifically targeting the carotid artery. The Doppler Ultrasound Transducer Device 5 on the Doppler ultrasound patch 7 may be secured in place with a fixation mechanism in the region of the subject's neck 8, ensuring stable and hands-free operation during CPR. In at least some embodiments, the fixation mechanism may be an adhesive patch. Accordingly, this configuration may allow the Doppler Ultrasound Transducer Device 5 to emit continuous-wave ultrasound signals and receive Doppler return signals indicative of blood flow patterns in the carotid artery.

In at least some embodiments, the Doppler return signals may be wirelessly transmitted to the processing unit 20, which is configured to analyze the signals in real time. The processing unit 20 may employ advanced algorithms, such as a Doppler-Integrated Cardiac Arrest Flow Analysis (DICAF) Algorithm, to distinguish between bidirectional or minimal blood flow generated by chest compressions 15 and blood flow patterns indicative of cardiac activity originating from the heart itself during chest compressions. As a result, this analysis may enable the detection of ROSC-specific patterns, such as superimposed systolic pulses or anterograde-dominant flow.

In at least some embodiments, the results of the analysis may be displayed on a display 25 on the processing unit 20, which provides actionable feedback to the user 17 performing CPR. The display 25 may indicate whether ROSC has been detected or whether chest compressions 15 are to continue due to the absence of ROSC. This real-time feedback may help reduce interruptions in chest compressions 15 and improve the quality of resuscitation efforts.

In at least some embodiments, the processing unit 20 may be a mobile computing device such as a tablet or a smartphone.

In at least some embodiments, the processing unit 20 may be part of a defibrillation device, or a lifesaving device.

In at least some embodiments, the user 17 performing chest compressions 15 may rely on the illustrative system's feedback to optimize the CPR technique, ensuring effective blood flow generation and timely recognition of ROSC. Moreover, the hands-free nature of the Doppler ultrasound patch 7 and the automated analysis performed by the processing unit 20 may reduce the need for manual pulse checks, thereby improving the chest compression fraction and overall resuscitation efficacy.

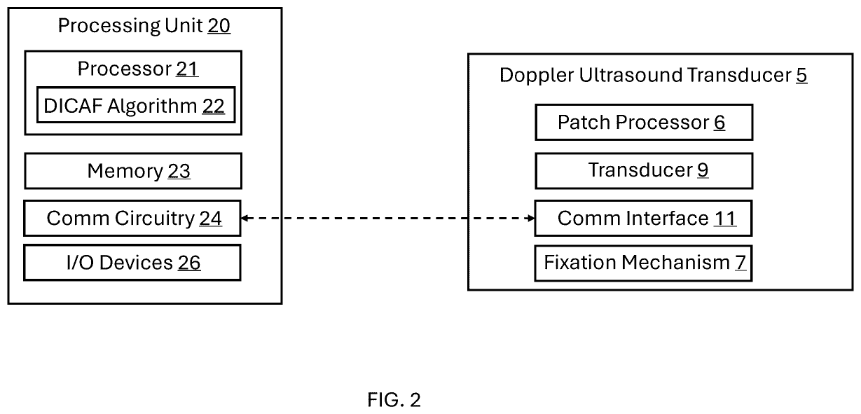

FIG. 2 is a block diagram illustrating an embodiment of a processing system 200 for real-time analysis of Doppler ultrasound signals to detect return of spontaneous circulation (ROSC) in accordance with one or more embodiments of the present disclosure. In at least some embodiments, the processing system 200 may include a processing unit 20 that serves as the central component for analyzing Doppler ultrasound signals received from a Doppler ultrasound transducer device 5.

In at least some embodiments, the processing unit 20 may comprise a processor 21 configured to execute instructions stored in a memory 23. The memory 23 may contain a Doppler-Integrated Cardiac Arrest Flow Analysis (DICAF) algorithm 22, which is specifically designed to analyze Doppler signals and distinguish between blood flow patterns generated by chest compressions and those indicative of cardiac activity originating from the heart itself. Accordingly, the DICAF algorithm 22 enables the processing unit 20 to identify ROSC-specific patterns—such as superimposed systolic pulses or anterograde-dominant flow—in real time.

In at least some embodiments, the processing unit 20 may further include communication circuitry 24, which facilitates wireless data exchange with the Doppler ultrasound transducer device 5. This circuitry supports the transmission of Doppler return signals for analysis and allows the processing unit 20 to send alerts or recommendations to input/output (I/O) devices 26. The I/O devices 26 may include, but are not limited to, visual displays, audible alarms, or haptic feedback mechanisms to provide actionable feedback to users performing CPR.

In at least some embodiments, the Doppler ultrasound transducer device 5 may be equipped with a transducer 9 that emits pulsed wave or continuous-wave ultrasound signals and receives Doppler return signals indicative of blood flow in the carotid, femoral, or brachial artery. The transducer 9 may be integrated with a patch processor 6, which performs preliminary signal processing before transmitting the data to the processing unit 20. The patch processor 6 prepares the Doppler signals for further analysis by the DICAF algorithm 22.

In at least some embodiments, the Doppler ultrasound transducer device 5 may also include a communication interface 11, which enables wireless transmission of processed signals to the processing unit 20. This interface may support short-range wireless protocols such as Bluetooth or Wi-Fi, thereby ensuring reliable and efficient data transfer during ongoing chest compressions. Additionally, the transducer device 5 may be secured in place using a fixation mechanism 7, such as an adhesive patch, to maintain stable positioning over the carotid artery during CPR. This hands-free configuration facilitates continuous monitoring and removes the requirement for manual adjustments.

Accordingly, by integrating the processing unit 20 and the Doppler ultrasound transducer device 5, the illustrative system provides real-time, non-invasive monitoring of blood flow during cardiac arrest. Moreover, by leveraging the DICAF algorithm 22 and advanced communication capabilities, the illustrative system provides immediate feedback on ROSC status, thereby optimizing CPR quality and reducing delays in post-resuscitation care.

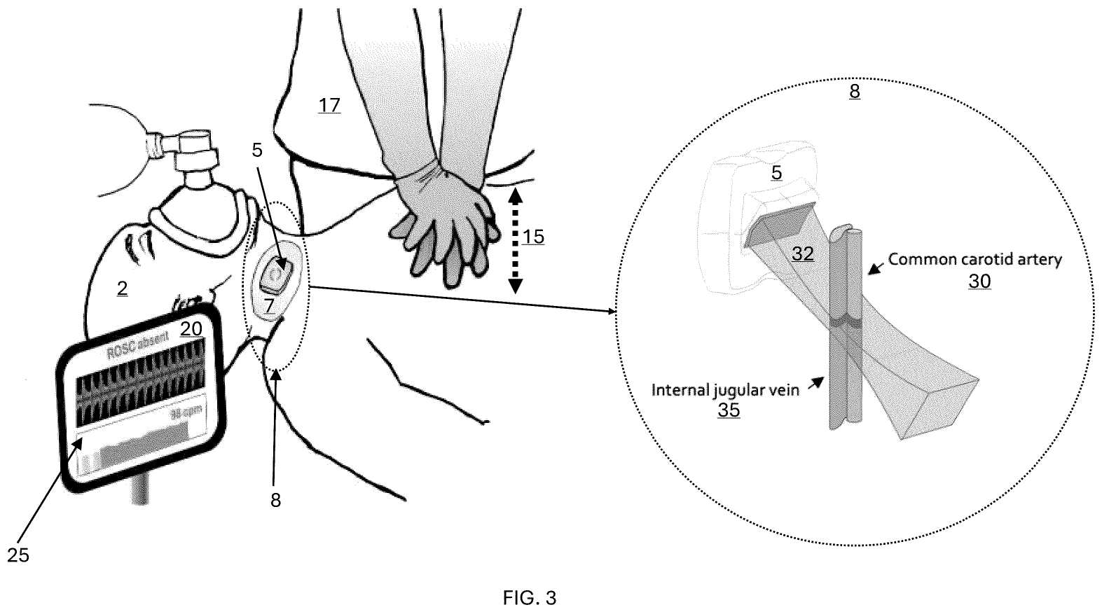

FIG. 3 is a biological diagram illustrating CPR with Doppler ultrasound analysis, showing real-time monitoring of blood flow in the carotid artery using a wearable patch 7 and processing unit 20 in accordance with one or more embodiments of the present disclosure. In at least some embodiments, a Doppler Ultrasound Transducer Device 5 is secured to the subject's neck region 8 via an adhesive patch 7 to facilitate hands-free operation during chest compressions 15.

In at least some embodiments, a subject 2 undergoing CPR performed by a user 17 may receive chest compressions 15 applied to the subject's chest to generate circulatory flow during cardiac arrest. The Doppler Ultrasound Transducer Device 5, positioned over the common carotid artery 30 and internal jugular vein 35, emits a wide continuous wave (CW) ultrasound beam 32 and receives Doppler return signals indicative of bidirectional and anterograde blood flow patterns. This configuration ensures stable placement and uninterrupted insonation of the target vessels even during vigorous compressions, allowing for the differentiation of compression-induced flow from cardiac activity generated by the heart itself.

In at least some embodiments, the Doppler return signals are wirelessly transmitted to a processing unit 20 for real-time analysis. The processing unit 20 may employ advanced signal-processing algorithms, such as the Doppler-Integrated Cardiac Arrest Flow Analysis (DICAF) Algorithm, to detect return of spontaneous circulation (ROSC)-specific patterns, including superimposed systolic pulses or anterograde-dominant flow. Accordingly, the results of this analysis are displayed on a display 25, providing actionable feedback to the user 17 regarding whether ROSC has been detected or whether chest compressions 15 are to be continued in the absence of ROSC.

In at least some embodiments, this real-time feedback mechanism enables optimization by recognizing an amount of blood flow generated by CPR technique and/or providing feedback to alert the team to minimal blood flow to alter CPR, thereby improving chest compression quality and fraction, as well as overall resuscitation efficacy. Note that by leveraging the hands-free nature of the adhesive patch 7 and the automated analysis performed by the processing unit 20, the illustrative system reduces or eliminates the need for manual pulse checks. As a result, clinicians and rescuers can maintain consistent compression depth and rate while obtaining timely recognition of ROSC, as contemplated by the embodiments herein disclosed.

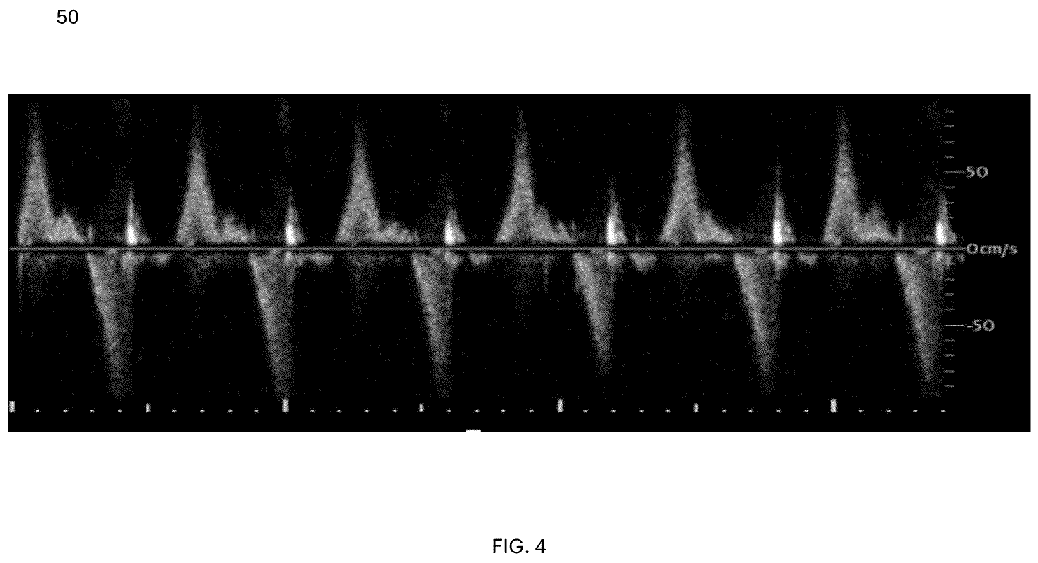

FIG. 4 is a graph illustrating a Doppler blood flow waveform 50 showing bidirectional flow during cardiac arrest in accordance with one or more embodiments of the present disclosure. In at least some embodiments, a Doppler ultrasound system 5 may include a Doppler ultrasound patch 7 and a processing unit 20 for implementing real-time waveform analysis in a service layer as described hereinbelow. In at least some embodiments, at least one flow analyzer software module may be configured to detect and characterize the bidirectional blood flow waveform 50 in response to chest compressions 15 during cardiopulmonary resuscitation (CPR).

In at least some embodiments, the bidirectional blood flow waveform 50 may demonstrate alternating anterograde and retrograde flow components, which are indicative of blood flow induced by external chest compressions 15 in the absence of spontaneous cardiac activity. The anterograde flow component corresponds to the forward movement of blood during the compression phase, while the retrograde flow component corresponds to the backward movement of blood during the decompression phase. Note that this bidirectional pattern is a hallmark of mechanical blood flow generated by CPR and is distinguishable from the unidirectional flow typically associated with natural cardiac function.

Accordingly, in at least some embodiments, the Doppler ultrasound system 5, which includes the Doppler ultrasound patch 7 and the processing unit 20, may be configured to detect and analyze such bidirectional waveforms in real time. The processing unit 20 may employ the Doppler-Integrated Cardiac Arrest Flow Analysis (DICAF) Algorithm 22 to identify the bidirectional flow pattern and to distinguish it from other blood flow patterns that signify cardiac activity originating from the heart, such as superimposed systolic pulses or anterograde-dominant flow.

In at least some embodiments, the bidirectional blood flow waveform 50 serves as a baseline reference for the illustrative system to assess the performance of chest compressions 15 and to identify the return of spontaneous circulation (ROSC). For instance, the illustrative system may observe variations in the waveform—such as the appearance of superimposed systolic pulses or a transition toward anterograde-dominant flow—to recognize the initiation of cardiac activity during ongoing chest compressions 15.

In at least some embodiments, by continuously monitoring and analyzing the Doppler blood flow waveform 50, the illustrative system provides real-time feedback to rescuers, allowing them to refine CPR quality and reduce interruptions. This capability increases the chances of achieving ROSC and supports better resuscitation outcomes.

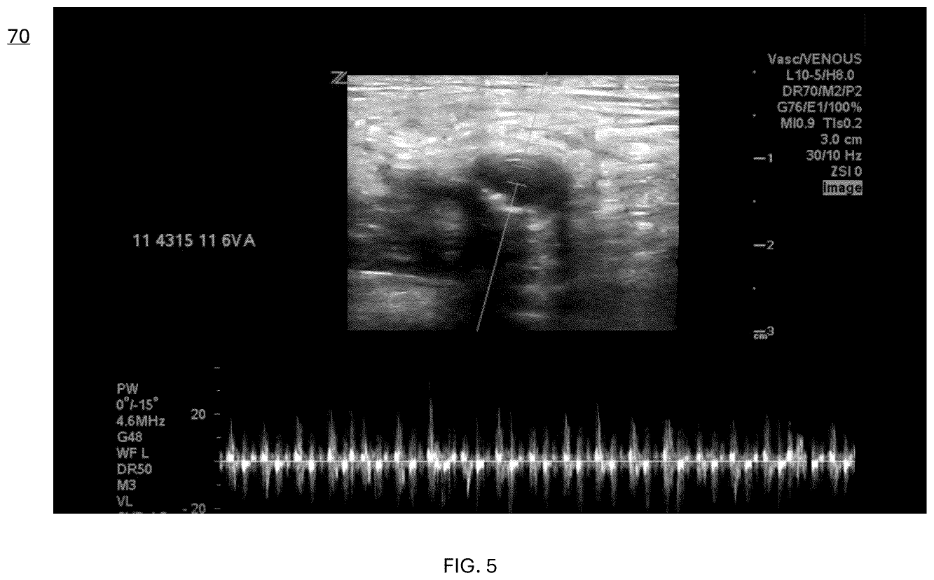

FIG. 5 is an ultrasound image illustrating a Doppler waveform depicting pulsatility through chest compressions 70 in accordance with one or more embodiments of the present disclosure. In at least some embodiments, a Doppler ultrasound monitoring system may include a transducer module, a signal processing module, and a display interface for rendering real-time Doppler waveforms as described hereinbelow. In at least some embodiments, at least one waveform analysis software module may be configured to capture and analyze pulsatility 70 in response to chest compressions 15 administered to a subject 2 during cardiopulmonary resuscitation (CPR).

In at least some embodiments, the pulsatility 70 shown in the Doppler waveform may represent dynamic blood flow patterns generated during chest compressions 15 via periodic oscillations that reflect the mechanical effects of external compressions on the carotid artery 30. Accordingly, the Doppler waveform captures both anterograde and retrograde flow components—indicative of bidirectional blood flow 50 caused by the compression and decompression phases of CPR—and these oscillatory patterns serve as a baseline for assessing the quality of chest compressions 15 and detecting cardiac activity originating from within the heart.

In at least some embodiments, the Doppler waveform may exhibit superimposed systolic pulses, which are indicative of cardiac contractility during ongoing chest compressions 15. These systolic pulses are distinguishable from the bidirectional flow 50 generated solely by external compressions and may signify the return of spontaneous circulation (ROSC). In at least some embodiments, the illustrative system may utilize advanced algorithms, such as the Doppler-Integrated Cardiac Arrest Flow Analysis (DICAF) Algorithm 22, to identify ROSC-specific patterns in real time.

In at least some embodiments, the waveform may transition to an anterograde-dominant flow pattern 100, where the forward flow component becomes more pronounced relative to the retrograde flow. This transition may occur when cardiac activity begins to contribute to blood flow, even if individual systolic pulses are not visually apparent. The detection of such anterograde-dominant flow 100 is another indicator of ROSC and may be used by the illustrative system to provide actionable feedback to rescuers without requiring interruptions for manual pulse checks. By leveraging real-time Doppler data, the illustrative system improves the precision and responsiveness of ROSC detection, thereby enhancing resuscitation efforts and supporting better patient outcomes.

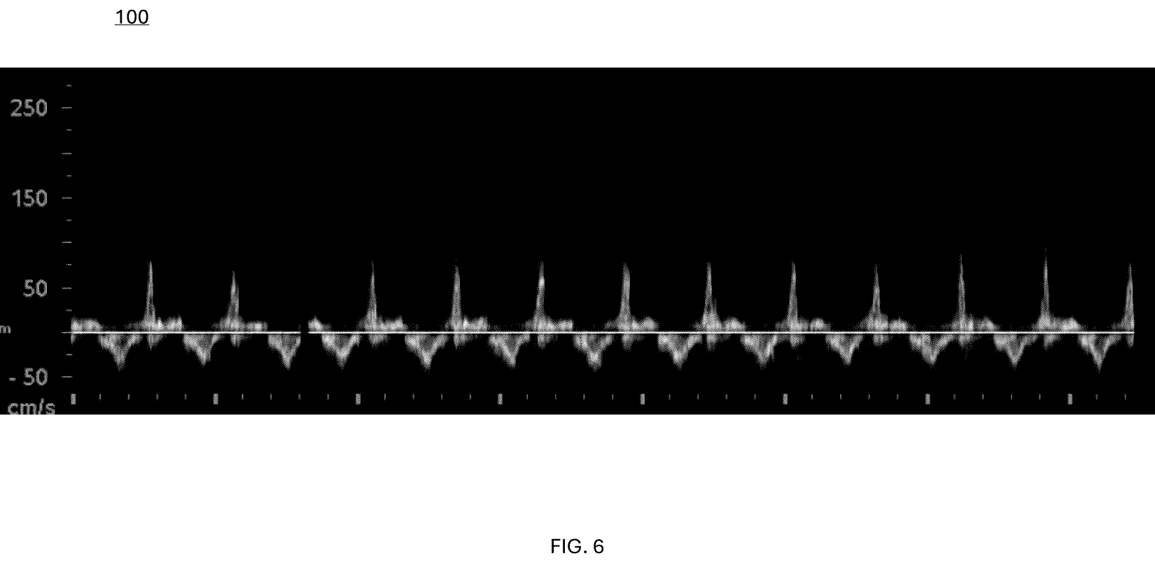

FIG. 6 is a waveform diagram illustrating anterograde-dominant blood flow 100 during chest compressions in accordance with one or more embodiments of the present disclosure. In at least some embodiments, a resuscitation monitoring system may include a Doppler ultrasound transducer device 5, a signal acquisition module, and a processing unit 20 for implementing the Doppler-Integrated Cardiac Arrest Flow Analysis (DICAF) Algorithm 22 in a system layer as described hereinbelow. In at least some embodiments, at least one waveform analyzer software module may be configured to detect anterograde-dominant blood flow 100 in response to chest compressions 15 and cardiac activity as represented by the waveform 100.

In at least some embodiments, the anterograde-dominant blood flow pattern 100 is detected using a Doppler ultrasound system that continuously monitors the common carotid artery 30 during cardiac arrest. The Doppler waveform analysis module may reveal that the forward flow component becomes more pronounced, as evidenced by the higher amplitude of the anterograde peaks compared to the retrograde troughs. Accordingly, this shift in flow dynamics may occur as the contractile forces of the heart begin to supplement or override the mechanical flow induced by chest compressions 15.

In at least some embodiments, the detection of anterograde-dominant blood flow 100 is facilitated by the DICAF Algorithm 22 implemented in the processing unit 20 of the illustrative system. The DICAF Algorithm 22 may analyze Doppler return signals in real time to identify this specific flow pattern. Note that bidirectional flow patterns 50 generated solely by external compressions 15 do not exhibit the characteristic predominance of anterograde flow 100, which is indicative of the physiological contribution of the heart even in the absence of visually distinct systolic pulses.

In at least some embodiments, the illustrative system may use this waveform pattern 100 as a reliable indicator of return of spontaneous circulation (ROSC). The predominance of anterograde flow 100 may suggest that the heart is effectively propelling blood forward, thereby reducing the retrograde component typically associated with the decompression phase of chest compressions 15. This transition in flow dynamics may take place prior to the appearance of distinct systolic pulses, offering an initial marker for ROSC.

In at least some embodiments, the real-time detection of anterograde-dominant blood flow 100 offers notable benefits for resuscitation efforts. By identifying this pattern, the illustrative system may provide actionable feedback to rescuers, for example by recommending adjustments to chest compression technique 15 or signaling the need to transition to post-resuscitation care. Interruptions in chest compressions 15 for manual pulse checks may be eliminated, which can improve the chest compression fraction and enhance the overall effectiveness of cardiopulmonary resuscitation (CPR).

In at least some embodiments, the waveform analyzer software module may also assess the quality of chest compressions 15. The illustrative system may analyze parameters such as the velocity-time integral measurement (VTM) and peak systolic velocity to evaluate the effectiveness of compressions 15 in generating forward blood flow 100. Accordingly, this physiologically guided feedback enables rescuers to refine compression depth, rate, and location, adapting the resuscitation process to the individual subject's 2 needs.

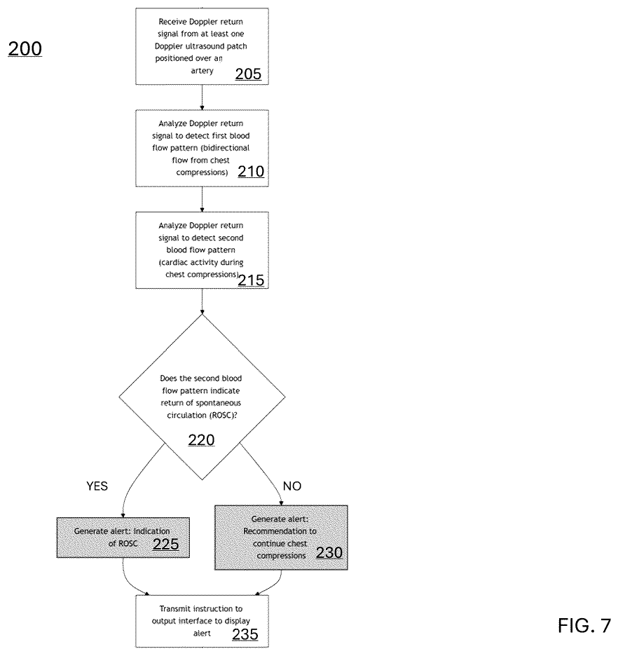

FIG. 7 is a flowchart illustrating a method 200 for Doppler signal analysis to detect return of spontaneous circulation (ROSC) 220 and generate corresponding alerts 225, 230 in accordance with one or more embodiments of the present disclosure. The method 200 may be performed by the processor 21. In other embodiments, the method 200 may be performed by the patch processor 6.

In at least some embodiments, the method 200 may be applied in systems utilizing at least one Doppler ultrasound transducer 5 on a patch 7 to continuously monitor hemodynamic performance during cardiopulmonary resuscitation (CPR).

In at least some embodiments, the method 200 may include receiving Doppler return signals 205 from at least one Doppler ultrasound patch positioned over an artery, or a selected artery such as a carotid artery, a femoral, or brachial artery. The Doppler ultrasound patch 5,7 may be configured to continuously monitor blood flow in the selected artery during ongoing chest compressions, ensuring that real-time data indicative of blood flow patterns is captured and transmitted to a processing unit 20 for further analysis.

In at least some embodiments, the method 200 may include analyzing the Doppler return signals to detect a first blood flow pattern 210, which may correspond to bidirectional flow generated by mechanical chest compressions. Bidirectional flow may be characterized by alternating anterograde and retrograde components, reflecting the mechanical effects of external compressions 15 on the selected artery. This analysis may serve as a baseline for distinguishing compression-induced flow from cardiac activity originating from the heart itself.

In at least some embodiments, the method 200 may include analyzing the Doppler return signals to detect a second blood flow pattern 215, which corresponds to cardiac activity generated during ongoing chest compressions 15. The second blood flow pattern may include features such as superimposed systolic pulses or anterograde-dominant flow, which may be indicative of cardiac contributions to blood flow.

In at least some embodiments, advanced algorithms—such as the Doppler-Integrated Cardiac Arrest Flow Analysis (DICAF) Algorithm 22—may be leveraged to identify ROSC-specific patterns.

In at least some embodiments, the method 200 may include evaluating 220 whether the detected second blood flow pattern 215 indicates a return of spontaneous circulation (ROSC). If ROSC is detected (e.g., “YES”), the illustrative system may generate a ROSC alert 225 indicating the occurrence of spontaneous circulation. This alert provides actionable feedback to the user 17, supporting recognition of ROSC and aiding the transition to post-resuscitation care.

In at least some embodiments, if the second blood flow pattern 215 does not indicate ROSC in the evaluating step 220 (e.g., “NO”), the illustrative system may generate an alert recommending the continuation of chest compressions 15. Accordingly, this may ensure that resuscitation efforts are maintained without unnecessary interruptions, thereby optimizing chest compression fraction and improving the likelihood of achieving ROSC 220.

In at least some embodiments, the method 200 may include transmitting 235 instructions to an output interface 26 for displaying the generated alert 225, 230. The output interface 26 may include visual displays 25, audible alarms, or haptic feedback mechanisms, providing real-time feedback to rescuers performing CPR. This step may facilitate the communication of actionable information to the user 17, aiding in physiologically guided resuscitation and improving CPR performance.

In at least some embodiments, a system may include a Doppler ultrasound patch and a processing unit that may provide real-time feedback to guide the location of chest compressions during cardiopulmonary resuscitation (CPR). The illustrative system may analyze Doppler-derived blood flow parameters and may recommend adjustment of the compression site, such as moving compressions left of the sternum, to optimize hemodynamic outcomes including peak systolic velocity, velocity time integral, and flow time. The illustrative system may display actionable guidance to the user based on physiologic data, allowing for personalized CPR tailored to the individual patient's anatomy and response.

In at least some embodiments, the illustrative system may include functionality to create, curate, and utilize annotated datasets of Doppler ultrasound waveforms from the selected artery collected from both cardiac arrest and non-arrest subjects. These datasets may be used to train and validate machine learning algorithms for detection of return of spontaneous circulation (ROSC) and assessment of CPR quality. The illustrative system may support the ongoing expansion and refinement of these datasets to improve algorithmic performance and clinical reliability.

In at least some embodiments, the illustrative system may include algorithms that may assess the efficacy of chest compressions in real time by quantifying Doppler-derived metrics such as peak systolic velocity, velocity time integral, and flow time. The illustrative system may provide quantitative feedback to the user regarding the effectiveness of compressions and may recommend adjustments to compression depth, rate, or location to improve blood flow and resuscitation outcomes.

In at least some embodiments, the illustrative system may include automated methods for eliminating the need for manual pulse checks during CPR. The illustrative system may continuously analyze Doppler return signals to detect ROSC during ongoing chest compressions and may generate alerts indicating the presence or absence of spontaneous circulation, thereby allowing uninterrupted compressions and reducing delays in post-resuscitation care.

In at least some embodiments, the illustrative system may include a wide-beam continuous wave Doppler ultrasound patch that may facilitate reliable insonation of the selected artery such as but not limited to, for example, a carotid, a femoral, or a brachial artery regardless of minor placement variations. The illustrative system may be configured to operate with commercially available Doppler ultrasound devices, such as a FloPatch or Rescue Doppler, and may communicate wirelessly with external processing units or display devices. The illustrative system may also include comparative analysis modules that may evaluate the performance of the disclosed methods relative to prior art techniques for ROSC detection and CPR guidance.

In at least some embodiments, the illustrative system may include a method or device that may enable the personalization of cardiopulmonary resuscitation (CPR) by allowing a user to adjust the location of chest compressions, such as moving compressions left of the sternum, and may monitor for improvements in Doppler ultrasound blood flow parameters in real time to optimize hemodynamic outcomes, including systolic blood pressure.

In at least some embodiments, the illustrative system may include a method or device that may provide actionable feedback to the user regarding the effectiveness of chest compressions and may recommend adjustments to compression location, depth, or rate based on Doppler-derived blood flow metrics, such as peak systolic velocity, velocity time integral, or flow time, to improve CPR quality and patient outcomes.

In at least some embodiments, the illustrative system may include a method or device that may create, curate, and utilize annotated datasets of Doppler ultrasound waveforms from the selected artery collected from both cardiac arrest and non-arrest subjects, and may use these datasets to train and validate machine learning algorithms for detection of return of spontaneous circulation (ROSC) and assessment of CPR quality.

In at least some embodiments, the illustrative system may include a method or device that may provide a validated algorithm trained on waveform analysis data with machine learning from non-cardiac arrest waveforms in the selected artery and chest compression waveforms, and may use this algorithm to recognize blood flow patterns and calculate the probability that a patient has achieved ROSC.

In at least some embodiments, the illustrative system may include a method or device that may identify and analyze blood flow patterns from the preceding chest compression interval that may be associated with or without subsequent ROSC, and may use this information to improve the accuracy of ROSC detection.

In at least some embodiments, the illustrative system may include a method or device that may provide a dataset comprising Doppler ultrasound carotid, femoral, or brachial waveforms for non-cardiac arrest subjects and for subjects undergoing CPR, and may use this dataset to train artificial intelligence and machine learning algorithms for improved detection of ROSC and assessment of CPR quality.

In at least some embodiments, the illustrative system may include a method or device that may provide an automated tool for real-time analysis of CPR quality, and may allow providers to adjust manual CPR technique or the positioning and settings of an automated chest compression system to provide better results based on Doppler ultrasound feedback.

In at least some embodiments, the illustrative system may include a method or device that may provide a wearable Doppler ultrasound patch that may be applied easily to the skin over the selected artery, may be held in place with adhesive straps, and may continuously monitor blood flow to the brain, allowing for hands-free operation and reducing the need for specialized sonography training or additional staff members.

In at least some embodiments, an illustrative system may include at least one Doppler ultrasound patch including a continuous wave ultrasound transducer that may be configured to emit an ultrasound beam into a selected artery of a subject and may be configured to receive at least one Doppler return signal indicative of blood flow in the selected artery. The system may further include a fixation mechanism that may be configured to secure the continuous wave ultrasound transducer in a fixed position relative to the selected artery, and a wireless communication interface that may be configured to transmit the at least one Doppler return signal to a processing unit. The processing unit may include at least one transitory memory storing at least one instruction and at least one processor, which may be configured to execute the at least one instruction that may cause the at least one processor to receive the at least one Doppler return signal from the at least one Doppler ultrasound patch, analyze the at least one Doppler return signal indicative of the blood flow in the selected artery using a Doppler-Integrated Cardiac Arrest Flow Analysis (DICAF) algorithm to detect during ongoing chest compressions a first blood flow pattern including a bidirectional flow corresponding to the blood flow generated by the ongoing chest compressions and a second blood flow pattern corresponding to intrinsic cardiac activity originating from within a heart of the subject. The DICAF algorithm may include at least one machine learning model trained on annotated Doppler waveform datasets to distinguish between the blood flow generated by the ongoing chest compressions and the blood flow associated with the intrinsic cardiac activity in the first blood flow pattern and the second blood flow pattern. The second blood flow pattern may include at least one of systolic pulses generated by the heart superimposed on the first blood flow pattern or a predominance of an anterograde flow level relative to a retrograde flow level. The processing unit may determine in real time during the ongoing chest compressions a return of spontaneous circulation based on detection of the second blood flow pattern and may generate an alert including an indication of the return of spontaneous circulation or a recommendation to continue applying the ongoing chest compressions to the subject due to a lack of the return of spontaneous circulation, and may include an output interface to display the alert to a user.

In at least some embodiments, the at least one processor may be further configured to detect, using the DICAF algorithm, a transition from the first blood flow pattern to the second blood flow pattern during the ongoing chest compressions and may generate a time-stamped record of the transition.

In at least some embodiments, the fixation mechanism may include an adhesive patch that may be configured to maintain the continuous wave ultrasound transducer in a hands-free position over the selected artery.

In at least some embodiments, the wireless communication interface may include a Bluetooth, Wi-Fi, or other short-range wireless protocol for transmitting the at least one Doppler return signal to the processing unit. Alternatively, in at least other embodiments, this could also be connected by a wire to a defibrillator or other ultrasound device.

In at least some embodiments, the at least one processor may be configured to retrain the at least one machine learning model with updated annotated Doppler waveform datasets from a plurality of other subjects to improve an accuracy in detecting the return of spontaneous circulation in the subject.

In at least some embodiments, the output interface may include a visual display, an audible alarm, or a haptic feedback device to alert the user of the return of spontaneous circulation or a need to continue the ongoing chest compressions.

In at least some embodiments, the processing unit may be further configured to store Doppler return signals and analysis results in a plurality of electronic medical records.

In at least some embodiments, the at least one Doppler ultrasound patch may include a wide-beam continuous wave transducer to facilitate reliable insonation of the selected artery regardless of minor placement variations.

In at least some embodiments, the at least one processor may be configured to assess a quality of the ongoing chest compressions by analyzing Doppler-derived parameters such as peak systolic velocity, velocity time integral, flow time, or any combination thereof.

In at least some embodiments, the illustrative system may be configured to provide real-time feedback to guide adjustment of chest compression location or technique based on Doppler signal analysis.

In at least some embodiments, the selected artery may be a carotid artery, a femoral artery, or a brachial artery of the subject.

In at least some embodiments, a method may include receiving, by at least one processor, at least one Doppler return signal from at least one Doppler ultrasound patch positioned over a selected artery of a subject. The method may further include analyzing, by the at least one processor, the at least one Doppler return signal indicative of blood flow in the selected artery such as a carotid artery, a femoral artery, or brachial artery using a Doppler-Integrated Cardiac Arrest Flow Analysis (DICAF) algorithm to detect during ongoing chest compressions a first blood flow pattern including a bidirectional flow corresponding to the blood flow generated by the ongoing chest compressions and a second blood flow pattern corresponding to intrinsic cardiac activity originating from within a heart of the subject. The DICAF algorithm may include at least one machine learning model trained on annotated Doppler waveform datasets to distinguish between the blood flow generated by the ongoing chest compressions and the blood flow associated with the intrinsic cardiac activity in the first blood flow pattern and the second blood flow pattern. The second blood flow pattern may include at least one of systolic pulses generated by the heart superimposed on the first blood flow pattern or a predominance of an anterograde flow value relative to a retrograde flow value. The method may further include determining, by the at least one processor, in real time during the ongoing chest compressions, a return of spontaneous circulation based on detection of the second blood flow pattern, generating, by the at least one processor, an alert including an indication of the return of spontaneous circulation or a recommendation to continue applying the ongoing chest compressions to the subject due to a lack of the return of spontaneous circulation, and transmitting, by the at least one processor, at least one instruction to an output interface to display the alert (e.g., to a user applying the CPR to the subject).

In at least some embodiments, the selected artery may be a carotid artery, a femoral artery, a brachial artery of the subject.

In at least some embodiments, the analyzing of the at least one Doppler return signal may include detecting, using the DICAF algorithm, a transition from the first blood flow pattern to the second blood flow pattern during the ongoing chest compressions and may generate a time-stamped record of the transition.

In at least some embodiments, the at least one Doppler ultrasound patch may be secured in a hands-free position over the selected artery using an adhesive patch.

In at least some embodiments, transmitting the at least one Doppler return signal to the at least one processor may be performed using a Bluetooth, Wi-Fi, or other short-range wireless protocol.

In at least some embodiments, the method may further include retraining, by the at least one processor, the at least one machine learning model with updated annotated Doppler waveform datasets from a plurality of other subjects to improve an accuracy in detecting the return of spontaneous circulation in the subject.

In at least some embodiments, generating the alert may include providing a visual display, an audible alarm, or a haptic feedback to alert a user of the return of spontaneous circulation or a need to continue the ongoing chest compressions.

In at least some embodiments, the method may further include storing, by the at least one processor, analysis results and the at least one Doppler return signal in a plurality of electronic medical records.

In at least some embodiments, the at least one Doppler ultrasound patch may include a wide-beam continuous wave transducer to facilitate reliable insonation of the selected artery regardless of minor placement variations.

In at least some embodiments, the method may further include assessing, by the at least one processor, a quality of the ongoing chest compressions by analyzing Doppler-derived parameters such as peak systolic velocity, velocity time integral, flow time, or any combination thereof.

In at least some embodiments, the method may further include providing, by the at least one processor, real-time feedback to guide adjustment of chest compression location or technique based on Doppler signal analysis.

In at least some embodiments, the illustrative system may include a Doppler-Integrated Cardiac Arrest Flow Analysis (DICAF) Algorithm that may utilize advanced signal processing techniques to extract features from Doppler ultrasound waveforms. The DICAF Algorithm may analyze parameters such as peak systolic velocity, velocity time integral, and flow time, and may apply machine learning models—such as neural networks or random forests—that may be trained on annotated datasets of Doppler signals in a selected artery from both cardiac arrest and non-arrest subjects. The algorithm may be configured to continuously update its detection thresholds based on real-time data and may provide a probability score for the presence of return of spontaneous circulation (ROSC).

In at least some embodiments, the illustrative system may include a user interface that may display actionable feedback to rescuers in real time. The user interface may present visual indicators, such as color-coded alerts or waveform displays, and may provide audible or haptic feedback to guide the user in adjusting chest compression technique or location. The illustrative system may display specific recommendations, such as “move compressions left of sternum” or “increase compression depth,”based on the analysis of Doppler-derived blood flow parameters.

In at least some embodiments, the illustrative system may include a data storage module that may record Doppler return signals, analysis results, and user actions in a secure electronic medical record. The stored data may be used for retrospective analysis, quality assurance, or research purposes. The illustrative system may allow authorized users to review historical data, generate reports, and export anonymized datasets for further algorithm training and validation.

In at least some embodiments, the illustrative system may include safety features that may detect sensor dislodgement, signal loss, or device malfunction. The illustrative system may generate alerts to notify the user of any detected issues and may recommend corrective actions, such as repositioning the Doppler patch or checking wireless connectivity. The illustrative system may be designed to comply with relevant medical device safety standards and may include encryption and access controls to protect patient data.

In at least some embodiments, the illustrative system may include a multi-patch configuration, where two or more Doppler ultrasound patches may be applied to different vascular sites, such as bilateral carotid arteries or femoral arteries. The illustrative system may analyze signals from multiple sites to improve the accuracy of ROSC detection and may provide comparative feedback to guide optimal compression location and technique.

In at least some embodiments, the illustrative system may include a training mode that may simulate various clinical scenarios using prerecorded Doppler waveforms. The training mode may allow users to practice interpreting feedback and adjusting CPR technique in a controlled environment, thereby improving familiarity with the illustrative system and enhancing resuscitation skills.

In at least some embodiments, the illustrative system may include integration with automated chest compression devices. The illustrative system may communicate wirelessly with such devices and may provide real-time feedback to adjust compression rate, depth, or location based on Doppler signal analysis. The illustrative system may also support manual override by the user, allowing for personalized, physiologically guided resuscitation.

In at least some embodiments, the illustrative system may include a glossary or help module that may define key terms such as “bidirectional flow,” “minimal flow”, “anterograde-dominant flow,”, “pulsatility through compressions”, “real-time,” and “actionable feedback. ” The glossary may be accessible through the user interface and may provide visual examples and explanations to assist users in understanding the illustrative system's feedback and recommendations.

In at least some embodiments, the illustrative system may include a cloud-based analytics platform that may aggregate anonymized data from multiple devices and locations. The platform may support large-scale analysis of CPR quality and outcomes, may facilitate continuous improvement of the DICAF Algorithm, and may enable benchmarking of resuscitation performance across institutions.

In at least some embodiments, the illustrative system may include regulatory compliance features, such as audit trails, user authentication, and data encryption, to ensure adherence to healthcare privacy and security standards. The illustrative system may be designed to meet requirements for FDA clearance or CE marking, and may include documentation and labeling to support regulatory submissions.

In at least some embodiments, exemplary inventive, specially programmed computing systems/platforms with associated devices are configured to operate in the distributed network environment, communicating with one another over one or more suitable data communication networks (e.g., the Internet, satellite, etc.) and utilizing one or more suitable data communication protocols/modes such as the communication circuitry 24 and the communication interface 11 of FIG. 2, for example, such as, without limitation, IPX/SPX, X.25, AX.25, AppleTalk™, TCP/IP (e.g., HTTP), near-field wireless communication (NFC), RFID, Narrow Band Internet of Things (NBIOT), 3G, 4G, 5G, GSM, GPRS, WiFi, WiMax, CDMA, satellite, ZigBee, and other suitable communication modes. In at least some embodiments, the NFC can represent a short-range wireless communications technology in which NFC-enabled devices are “swiped,” “bumped,” “tap” or otherwise moved in close proximity to communicate. In at least some embodiments, the NFC could include a set of short-range wireless technologies, typically requiring a distance of 10 cm or less. In at least some embodiments, the NFC may operate at 13.56 MHz on ISO/IEC 18000-3 air interface and at rates ranging from 106 kbit/s to 424 kbit/s. In at least some embodiments, the NFC can involve an initiator and a target; the initiator actively generates an RF field that can power a passive target. In at least some embodiments, this can enable NFC targets to take very simple form factors such as tags, stickers, key fobs, or cards that do not require batteries. In at least some embodiments, the NFC's peer-to-peer communication can be conducted when a plurality of NFC-enable devices (e.g., smartphones) within close proximity of each other.

The material disclosed herein may be implemented in software or firmware or a combination of them or as instructions stored on a machine-readable medium, which may be read and executed by one or more processors. A machine-readable medium may include any medium and/or mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements as in FIG. 2, for example, may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In at least some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, as used herein, include any combination of hardware and software. Examples of software may include software components, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, etc.).

In at least some embodiments, one or more of exemplary inventive computer-based systems/platforms, exemplary inventive computer-based devices, and/or exemplary inventive computer-based components of the present disclosure may include or be incorporated, partially or entirely into at least one personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart tablet or smart television), mobile internet device (MID), messaging device, data communication device, and so forth.

As used herein, the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

In at least some embodiments, as detailed herein, one or more of exemplary inventive computer-based systems/platforms, exemplary inventive computer-based devices, and/or exemplary inventive computer-based components of the present disclosure may obtain, manipulate, transfer, store, transform, generate, and/or output any digital object and/or data unit (e.g., from inside and/or outside of a particular application) that can be in any suitable form such as, without limitation, a file, a contact, a task, an email, a social media post, a map, an entire application (e.g., a calculator), etc. In at least some embodiments, as detailed herein, one or more of exemplary inventive computer-based systems/platforms, exemplary inventive computer-based devices, and/or exemplary inventive computer-based components of the present disclosure may be implemented across one or more of various computer platforms such as, but not limited to: (1) FreeBSD, NetBSD, OpenBSD; (2) Linux; (3) Microsoft Windows; (4) OS X (MacOS); (5) MacOS 11; (6) Solaris; (7) Android; (8) iOS; (9) Embedded Linux; (10) Tizen; (11) WebOS; (12) IBM i; (13) IBM AIX; (14) Binary Runtime Environment for Wireless (BREW); (15) Cocoa (API); (16) Cocoa Touch; (17) Java Platforms; (18) JavaFX; (19) JavaFX Mobile; (20) Microsoft DirectX; (21) .NET Framework; (22) Silverlight; (23) Open Web Platform; (24) Oracle Database; (25) Qt; (26) Eclipse Rich Client Platform; (27) SAP NetWeaver; (28) Smartface; and/or (29) Windows Runtime.

In at least some embodiments, exemplary inventive computer-based systems/platforms, exemplary inventive computer-based devices, and/or exemplary inventive computer-based components of the present disclosure may be configured to utilize hardwired circuitry that may be used in place of or in combination with software instructions to implement features consistent with principles of the disclosure. Thus, implementations consistent with principles of the disclosure are not limited to any specific combination of hardware circuitry and software. For example, various embodiments may be embodied in many different ways as a software component such as, without limitation, a stand-alone software package, a combination of software packages, or it may be a software package incorporated as a “tool”in a larger software product.

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

In at least some embodiments, exemplary inventive computer-based systems/platforms, exemplary inventive computer-based devices, and/or exemplary inventive computer-based components of the present disclosure may be configured to handle numerous concurrent users that may be, but is not limited to, at least 100 (e.g., but not limited to, 100-999), at least 1,000 (e.g., but not limited to, 1,000-9,999), at least 10,000 (e.g., but not limited to, 10,000-99,999), at least 100,000 (e.g., but not limited to, 100,000-999,999), at least 1,000,000 (e.g., but not limited to, 1,000,000-9,999,999), at least 10,000,000 (e.g., but not limited to, 10,000,000-99,999,999), at least 100,000,000 (e.g., but not limited to, 100,000,000-999,999,999), at least 1,000,000,000 (e.g., but not limited to, 1,000,000,000-999,999,999,999), and so on.

In at least some embodiments, exemplary inventive computer-based systems/platforms, exemplary inventive computer-based devices, and/or exemplary inventive computer-based components of the present disclosure may be configured to output to distinct, specifically programmed graphical user interface implementations of the present disclosure (e.g., a desktop, a web app., etc.). In various implementations of the present disclosure, a final output may be displayed on a displaying screen which may be, without limitation, a screen of a computer, a screen of a mobile device, or the like. In various implementations, the display may be a holographic display. In various implementations, the display may be a transparent surface that may receive a visual projection. Such projections may convey various forms of information, images, and/or objects. For example, such projections may be a visual overlay for a mobile augmented reality (MAR) application.

As used herein, the term “mobile electronic device,” or the like, may refer to any portable electronic device that may or may not be enabled with location tracking functionality (e.g., MAC address, Internet Protocol (IP) address, or the like). For example, a mobile electronic device can include, but is not limited to, a mobile phone, a tablet, Personal Digital Assistant (PDA), Blackberry™, Pager, a Smartphone, or any other reasonable mobile electronic device.

As used herein, the terms “proximity detection,” “locating,” “location data,” “location information,” and “location tracking” refer to any form of location tracking technology or locating method that can be used to provide a location of, for example, a particular computing device/system/platform of the present disclosure and/or any associated computing devices, based at least in part on one or more of the following techniques/devices, without limitation: accelerometer(s), gyroscope(s), Global Positioning Systems (GPS); GPS accessed using Bluetooth™; GPS accessed using any reasonable form of wireless and/or non-wireless communication; WiFi™ server location data; Bluetooth ™ based location data; triangulation such as, but not limited to, network based triangulation, WiFi™ server information based triangulation, Bluetooth™ server information based triangulation; Cell Identification based triangulation, Enhanced Cell Identification based triangulation, Uplink-Time difference of arrival (U-TDOA) based triangulation, Time of arrival (TOA) based triangulation, Angle of arrival (AOA) based triangulation; techniques and systems using a geographic coordinate system such as, but not limited to, longitudinal and latitudinal based, geodesic height based, Cartesian coordinates based; Radio Frequency Identification such as, but not limited to, Long range RFID, Short range RFID; using any form of RFID tag such as, but not limited to active RFID tags, passive RFID tags, battery assisted passive RFID tags; or any other reasonable way to determine location. For ease, at times the above variations are not listed or are only partially listed; this is in no way meant to be a limitation.

As used herein, the terms “cloud,” “Internet cloud,” “cloud computing,” “cloud architecture,” and similar terms correspond to at least one of the following: (1) a large number of computers connected through a real-time communication network (e.g., Internet); (2) providing the ability to run a program or application on many connected computers (e.g., physical machines, virtual machines (VMs)) at the same time; (3) network-based services, which appear to be provided by real server hardware, and are in fact served up by virtual hardware (e.g., virtual servers), simulated by software running on one or more real machines (e.g., allowing to be moved around and scaled up (or down) on the fly without affecting the end user).

In at least some embodiments, the exemplary inventive computer-based systems/platforms, the exemplary inventive computer-based devices, and/or the exemplary inventive computer-based components of the present disclosure may be configured to securely store and/or transmit data by utilizing one or more of encryption techniques (e.g., private/public key pair, Triple Data Encryption Standard (3DES), block cipher algorithms (e.g., IDEA, RC2, RC5, CAST and Skipjack), cryptographic hash algorithms (e.g., MD5, RIPEMD-160, RTR0, SHA-1, SHA-2, Tiger (TTH), WHIRLPOOL, RNGs).

The aforementioned examples are, of course, illustrative and not restrictive.

As used herein, the term “user” shall have a meaning of at least one user. In at least some embodiments, the terms “user”, “subscriber” “consumer” or “customer” should be understood to refer to a user of an application or applications as described herein and/or a consumer of data supplied by a data provider. By way of example, and not limitation, the terms “user” or “subscriber” can refer to a person who receives data provided by the data or service provider over the Internet in a browser session, or can refer to an automated software application which receives the data and stores or processes the data.

In at least some embodiments, the exemplary inventive computer-based systems/platforms, the exemplary inventive computer-based devices, and/or the exemplary inventive computer-based components of the present disclosure (e.g., the DICAF Algorithm 22) may be configured to utilize one or more exemplary AI/machine learning techniques chosen from, but not limited to, decision trees, boosting, support-vector machines, neural networks, nearest neighbor algorithms, Naive Bayes, bagging, random forests, and the like. In some embodiments and, optionally, in combination of any embodiment described above or below, an exemplary neutral network technique may be one of, without limitation, feedforward neural network, radial basis function network, recurrent neural network, convolutional network (e.g., U-net) or other suitable network. In some embodiments and, optionally, in combination of any embodiment described above or below, an exemplary implementation of Neural Network may be executed as follows:
i) Define Neural Network architecture/model,
ii) Transfer the input data to the exemplary neural network model,
iii) Train the exemplary model incrementally,
iv) determine the accuracy for a specific number of timesteps,
v) apply the exemplary trained model to process the newly-received input data,
vi) optionally and in parallel, continue to train the exemplary trained model with a predetermined periodicity.

In at least some embodiments and, optionally, in combination of any embodiment described above or below, the exemplary trained neural network model may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below, the exemplary trained neural network model may also be specified to include other parameters, including but not limited to, bias values/functions and/or aggregation functions. For example, an activation function of a node may be a step function, sine function, continuous or piecewise linear function, sigmoid function, hyperbolic tangent function, or other type of mathematical function that represents a threshold at which the node is activated. In some embodiments and, optionally, in combination of any embodiment described above or below, the exemplary aggregation function may be a mathematical function that combines (e.g., sum, product, etc.) input signals to the node. In some embodiments and, optionally, in combination of any embodiment described above or below, an output of the exemplary aggregation function may be used as input to the exemplary activation function. In some embodiments and, optionally, in combination of any embodiment described above or below, the bias may be a constant value or function that may be used by the aggregation function and/or the activation function to make the node more or less likely to be activated.

In at least some embodiments, a method may include receiving, by at least one processor, at least one Doppler return signal from at least one Doppler ultrasound patch positioned over a carotid artery of a subject; analyzing, by the at least one processor, the at least one Doppler return signal to detect a first blood flow pattern that may include a bidirectional flow corresponding to blood flow generated by chest compressions; analyzing, by the at least one processor, the at least one Doppler return signal to detect a second blood flow pattern that may correspond to cardiac activity originating from within a heart of the subject during ongoing chest compressions, where the second blood flow pattern may include at least one of systolic pulses generated by the heart superimposed on the first blood flow pattern or a predominance of an anterograde flow value relative to a retrograde flow value; determining, by the at least one processor, in real time during the ongoing chest compressions, a return of spontaneous circulation based on detection of the second blood flow pattern; generating, by the at least one processor, an alert that may include an indication of the return of spontaneous circulation, or a recommendation to continue applying the ongoing chest compressions to the subject due to a lack of the return of spontaneous circulation; and transmitting, by the at least one processor, at least one instruction to an output interface to display the alert (e.g., to a user applying the CPR to the subject).

In at least some embodiments, the method may include analyzing the at least one Doppler return signal using a Doppler-Integrated Cardiac Arrest Flow Analysis (DICAF) Algorithm that may be configured to distinguish between the blood flow generated by the ongoing chest compressions and the blood flow generated by intrinsic cardiac activity.

In at least some embodiments, the method may include at least one Doppler ultrasound patch that may be secured in a hands-free position over the carotid artery using an adhesive patch.

In at least some embodiments, the method may include transmitting the at least one Doppler return signal to the at least one processor using a Bluetooth, Wi-Fi, or other short-range wireless protocol.

In at least some embodiments, the method may include analyzing the at least one Doppler return signal to detect the return of spontaneous circulation using a machine learning model that may be trained on annotated Doppler waveform datasets.

In at least some embodiments, the method may include generating the alert by providing a visual display, an audible alarm, or a haptic feedback to alert a user of the return of spontaneous circulation or a need to continue the ongoing chest compressions.

In at least some embodiments, the method may further include storing analysis results and the at least one Doppler return signal in a plurality of electronic medical records.

In at least some embodiments, the method may include at least one Doppler ultrasound patch that may include a wide-beam continuous wave transducer to facilitate reliable insonation of the carotid artery regardless of minor placement variations.

In at least some embodiments, the method may further include assessing a quality of the ongoing chest compressions by analyzing Doppler-derived parameters such as peak systolic velocity, velocity time integral, or flow time.

In at least some embodiments, the method may further include providing real-time feedback to guide adjustment of chest compression location or technique based on Doppler signal analysis.

Any publications cited throughout this document are hereby incorporated by reference in their entirety. While one or more embodiments of the present disclosure have been described, it is understood that these embodiments are illustrative only, and not restrictive, and that many modifications may become apparent to those of ordinary skill in the art, including that various embodiments of the inventive methodologies, the inventive systems/platforms, and the inventive devices described herein can be utilized in any combination with each other. Further still, the various steps may be carried out in any desired order (and any desired steps may be added and/or any desired steps may be eliminated).

1. An illustrative system, comprising:
at least one Doppler ultrasound patch comprising:
a continuous wave ultrasound transducer configured to emit an ultrasound beam into a selected artery of a subject and to receive at least one Doppler return signal indicative of blood flow in the selected artery;
a fixation mechanism configured to secure the continuous wave ultrasound transducer in a fixed position relative to the selected artery; and
a wireless communication interface configured to transmit the at least one Doppler return signal to a processing unit;
the processing unit, comprising at least one transitory memory storing at least one instruction and at least one processor, configured to execute the at least one instruction that causes the at least one processor to:
receive the at least one Doppler return signal from the at least one Doppler ultrasound patch;
analyze the at least one Doppler return signal indicative of the blood flow in the selected artery using a Doppler-Integrated Cardiac Arrest Flow Analysis (DICAF) algorithm to detect during ongoing chest compressions:
a first blood flow pattern comprising a bidirectional flow corresponding to the blood flow generated by the ongoing chest compressions and
a second blood flow pattern corresponding to intrinsic cardiac activity originating from within a heart of the subject;
wherein the DICAF algorithm comprises at least one machine learning model trained on annotated Doppler waveform datasets to distinguish between the blood flow generated by the ongoing chest compressions and the blood flow associated with the intrinsic cardiac activity in the first blood flow pattern and the second blood flow pattern;
wherein the second blood flow pattern comprises at least one of systolic pulses generated by the heart superimposed on the first blood flow pattern or a predominance of an anterograde flow level relative to a retrograde flow level;
determine in real time during the ongoing chest compressions, a return of spontaneous circulation based on detection of the second blood flow pattern; and
generate an alert comprising:
an indication of the return of spontaneous circulation, or
a recommendation to continue applying the ongoing chest compressions to the subject due to a lack of the return of spontaneous circulation; and
an output interface to display the alert.

2. The illustrative system of claim 1, wherein the at least one processor is further configured to detect, using the DICAF algorithm, a transition from the first blood flow pattern to the second blood flow pattern during the ongoing chest compressions, and to generate a time-stamped record of the transition.

3. The illustrative system of claim 1, wherein the fixation mechanism comprises an adhesive patch configured to maintain the continuous wave ultrasound transducer in a hands-free position over the selected artery.

4. The illustrative system of claim 1, wherein the wireless communication interface comprises a Bluetooth, Wi-Fi, or other short-range wireless protocol for transmitting the at least one Doppler return signal to the processing unit.

5. The illustrative system of claim 1, wherein the at least one processor is configured to retrain the at least one machine learning model with updated annotated Doppler waveform datasets from a plurality of other subjects to improve an accuracy in detecting the return of spontaneous circulation in the subject.

6. The illustrative system of claim 1, wherein the output interface comprises a visual display, an audible alarm, or a haptic feedback device to alert a user of the return of spontaneous circulation or a need to continue the ongoing chest compressions.

7. The illustrative system of claim 1, wherein the processing unit is further configured to store Doppler return signals and analysis results in a plurality of electronic medical records.

8. The illustrative system of claim 1, wherein the at least one Doppler ultrasound patch comprises a wide-beam continuous wave transducer to facilitate reliable insonation of the selected artery regardless of minor placement variations.

9. The illustrative system of claim 1, wherein the at least one processor is configured to assess a quality of the ongoing chest compressions by analyzing Doppler-derived parameters such as peak systolic velocity, velocity time integral, flow time, or any combination thereof.

10. The illustrative system of claim 1, wherein the illustrative system is configured to provide real-time feedback to guide adjustment of chest compression location or technique based on Doppler signal analysis.

11. The illustrative system of claim 1, wherein the selected artery is a carotid artery, a femoral artery, or a brachial artery.

12. A method, comprising:
receiving, by at least one processor, at least one Doppler return signal from at least one Doppler ultrasound patch positioned over a selected artery of a subject;
analyzing, by the at least one processor, the at least one Doppler return signal indicative of blood flow in the selected artery using a Doppler-Integrated Cardiac Arrest Flow Analysis (DICAF) algorithm to detect during ongoing chest compressions:
a first blood flow pattern comprising a bidirectional flow corresponding to the blood flow generated by the ongoing chest compressions and
a second blood flow pattern corresponding to intrinsic cardiac activity originating from within a heart of the subject;
wherein the DICAF algorithm comprises at least one machine learning model trained on annotated Doppler waveform datasets to distinguish between the blood flow generated by the ongoing chest compressions and the blood flow associated with the intrinsic cardiac activity in the first blood flow pattern and the second blood flow pattern;
wherein the second blood flow pattern comprises at least one of systolic pulses generated by the heart superimposed on the first blood flow pattern or a predominance of an anterograde flow value relative to a retrograde flow value;
determining, by the at least one processor, in real time during the ongoing chest compressions, a return of spontaneous circulation based on detection of the second blood flow pattern;
generating, by the at least one processor, an alert comprising:
an indication of the return of spontaneous circulation, or
a recommendation to continue applying the ongoing chest compressions to the subject due to a lack of the return of spontaneous circulation; and
transmitting, by the at least one processor, at least one instruction to an output interface to display the alert.

13. The method of claim 12, wherein the analyzing of the at least one Doppler return signal comprises detecting, using the DICAF algorithm, a transition from the first blood flow pattern to the second blood flow pattern during the ongoing chest compressions, and to generate a time-stamped record of the transition.

14. The method of claim 12, wherein the at least one Doppler ultrasound patch is secured in a hands-free position over the selected artery using an adhesive patch.

15. The method of claim 12, wherein transmitting the at least one Doppler return signal to the at least one processor is performed using a Bluetooth, Wi-Fi, or other short-range wireless protocol.

16. The method of claim 12, further comprising retraining, by the at least one processor, the at least one machine learning model with updated annotated Doppler waveform datasets from a plurality of other subjects to improve an accuracy in detecting the return of spontaneous circulation in the subject.

17. The method of claim 12, wherein generating the alert comprises providing a visual display, an audible alarm, or a haptic feedback to alert a user of the return of spontaneous circulation or a need to continue the ongoing chest compressions.

18. The method of claim 12, further comprising storing, by the at least one processor, analysis results and the at least one Doppler return signal in a plurality of electronic medical records.

19. The method of claim 12, wherein the at least one Doppler ultrasound patch comprises a wide-beam continuous wave transducer to facilitate reliable insonation of the selected artery regardless of minor placement variations.

20. The method of claim 12, further comprising assessing, by the at least one processor, a quality of the ongoing chest compressions by analyzing Doppler-derived parameters such as peak systolic velocity, velocity time integral, flow time, or any combination thereof.

21. The method of claim 12, further comprising providing, by the at least one processor, real-time feedback to guide adjustment of chest compression location or technique based on Doppler signal analysis.

22. The method of claim 12, wherein the selected artery is a carotid artery, a femoral artery, or a brachial artery.
